(19) United States
(12) Patent Application Publication
(10) Pub. No.: US 2007/0213671 A1
(43) Pub. Date: Sep. 13, 2007

(54) Infusion catheter system with telescoping cannula

(76) Inventor: Mark J. Hiatt, Ellettsville, IN (US)

Correspondence Address: BRINKS HOFER GILSON & LIONE/CHICAGO/COOK, PO BOX 10395, CHICAGO, IL, 60610, US

(21) Appl. No.: 11/515,705

(22) Filed: Sep. 5, 2006

Related U.S. Application Data
(60) Provisional application No. 60/715,441, filed on Sep. 7, 2005.

(51) Int. Cl. A61M 5/178

(52) U.S. Cl. 604/164.01

(57) ABSTRACT

An infusion catheter system includes an elongated catheter body and an inner elongated cannula body. The catheter body has a sidewall perforated with a plurality of side ports and the cannula body may have an outlet opening in a distal end. The side ports of the catheter body are selectively in fluid communication with the outlet opening of the cannula by moving the cannula between a first and second position within the catheter body.

### BACKGROUND OF THE INVENTION

The present invention relates generally to an apparatus and method for the delivery of therapeutic fluids to bodily passages and particularly infusion catheters exhibiting uniform distribution of such therapeutic fluids along the length of such passages.

In certain medical conditions, it is advantageous to deliver a therapeutic agent directly to a target region to avoid medicating the entire body and to limit the amount of therapeutic agent required for effective treatment. Alternatively, it may be advantageous to treat the entire region, but in a controlled and measured fashion. One example of such a medical condition is a varicose vein, which can be treated effectively by localized and uniform application of such therapeutic fluids along the length of the vessel, beginning at the uppermost region.

Infusion catheters have been developed which can deliver therapeutic fluids directly to affected bodily passages, for example a thrombotic region of an artery. One type of infusion catheter is a hollow tube, the distal end of which has been pierced through its side wall to form multiple openings, or ports, thereby providing for fluid communication between a central lumen and the exterior of the catheter. The ports are disposed at several axial positions along the infusion section to provide distribution of the therapeutic fluid along a desired length of the bodily passage. However, fluids flowing through a tube flow more readily from ports offering the least flow resistance. The longer the flow path followed by the fluid in the central lumen, the higher the resistance and the higher the pressure drop in the fluid. If the infusion section of the catheter has multiple ports or passageways, the fluid flowing from each port typically exhibits resistance and a pressure drop proportional to the distance of fluid flow along the length of the central lumen. Thus, fluid flowing to more distal ports experiences a higher pressure drop than fluid flowing to more proximal ports. As a result, the fluid distribution to the exterior of the catheter along the length of the catheter is not uniform.

It would be desirable to have an infusion catheter whose performance is not affected by the length of the inner lumen or the distance the therapeutic fluid travels between side ports. It would also be desirable to dispense fluid from the distal end of a catheter before dispensing fluid from the proximal end of the catheter.

### BRIEF SUMMARY OF THE INVENTION

The catheter described below may overcome the aforementioned problems and relates to a medical device, and more particularly, to an infusion catheter system that is capable of delivering a variety of fluids to designated areas of the human body at a controlled and uniform fluid discharge rate. The invention further relates to an infusion catheter system that is capable of delivering a variety of fluids to the human body. The invention further relates to a method for using such a catheter system and for treating varicose veins.

In accordance with the present invention, an infusion catheter system for the uniform delivery rate of a fluid is provided where the device comprises:

an elongated catheter body, the catheter body comprising a first distal end, a first proximal end, and at least a first lumen; the first lumen defined by a first outer sidewall extending between the distal end and the proximal end; the sidewall having a plurality of side ports therethrough, the side ports including at least a first side port and a second side port disposed longitudinally along the catheter body;

an elongated cannula body disposed within the catheter body and comprising a second distal end, a second proximal end, and a second lumen, the second lumen being defined by a second sidewall extending between the second distal end and second proximal end, and an outlet opening disposed through the cannula; and

wherein the cannula is movable along a longitudinal axis from a first position, wherein the outlet opening is primarily in fluid communication with the first side port, and a second position, wherein the outlet opening is primarily in fluid communication with said second side port.

The infusion catheter system, as described above, wherein the plurality of side ports each have an inner diameter of approximately 0.2 mm to approximately 2.0 mm.

The infusion catheter system as described above, wherein the plurality of side ports each have an inner diameter of approximately 0.8 mm.

The infusion catheter system, as described above, wherein the plurality of side ports each have an inner diameter of approximately 0.8 mm and wherein the side ports are longitudinally disposed along the side wall approximately every one cm over a 20 cm length.

The infusion catheter system, as described above, wherein the side ports are disposed longitudinally along the catheter body for at least 20 cm, and wherein the side ports are disposed approximately 1 cm from one another.

The infusion catheter system, as described above, wherein the catheter body further comprises a first radiopaque band, the band disposed circumferentially around the catheter body at the first distal end.

The infusion catheter system, as described above, wherein the catheter body further comprises a second radiopaque band, the band disposed circumferentially around the catheter body at the first proximal end; and wherein the first radiopaque band and the second radiopaque band delineate a boundary of the catheter body having the side ports.

The infusion catheter, as described above, wherein the system further comprises an occlusion device disposed distal of the first distal end.

The infusion catheter system, as described above, wherein the elongated cannula body is characterized by the lack of outlet openings in the second side wall and wherein the outlet opening is disposed through the second distal end of said cannula body.

The infusion catheter, as described above, wherein the outlet opening is disposed through the second sidewall, toward the second distal end.

A method of treating a varicose vein has been devised comprising the steps of:
inserting an infusion catheter system into a vein, the catheter system comprising an elongated catheter body, the catheter comprising a first distal end, a first proximal end, and at least a first lumen; the first lumen defined by a first outer sidewall extending between the first distal end and the first proximal end; the first outer sidewall having a plurality of side ports therethrough, the side ports including at least a first side port and a second side port disposed longitudinally along the catheter body; an elongated cannula body disposed within the catheter body and comprising a second distal end, a second proximal end, and an inner lumen, the inner lumen being defined by a second sidewall extending between the second distal end and second proximal end; an outlet opening disposed through the second distal end; and wherein the cannula is movable along a longitudinal axis from at least a first position, wherein the outlet opening is primarily in fluid communication with the first side port, and a second position, wherein the outlet opening is primarily in fluid communication with the second side port;
injecting a sclerosant into the cannula body, when the cannula body is in the first position, said sclerosant thereby passing through said first side port and sclerosing a first portion of the vein;
moving the cannula body from the first position to the second position;
injecting the sclerosant into the cannula body when the cannula body is in the second position, said sclerosant thereby passing through said second side port and sclerosing a second portion of said vein.

The method, as described above, wherein the method further comprises inserting a guidewire into the vein and manipulating the guidewire through the vein to reach a position to be treated.

The method, as described above, wherein the catheter system further comprises an occlusion balloon, the balloon being inflated around the first distal end of the catheter body before the sclerosant is injected into the cannula body.

The method, as described above, wherein the catheter system further comprises at least a first radiopaque marker disposed at the first distal end of the catheter body and defining the boundary of a portion of the catheter body having the side ports.

The method, as described above, wherein the catheter system further comprises a second radiopaque marker disposed at the first proximal end of the catheter body and further defining the boundary of the portion of the catheter body having the side ports.

The method, as described above, wherein the catheter system is positioned, using the boundary defined by the first radiopaque marker and the second radiopaque marker, to overlap an area of the vein to be treated and positioning the cannula body relative to the first radiopaque marker and the second radiopaque marker.

The method, as described above, wherein the plurality of side ports are arranged in a single row and are disposed along a longitudinal axis of the infusion catheter system.

The method, as described above, wherein the plurality of side ports are arranged in two rows along a longitudinal axis of the infusion catheter system.

The method, as described above, wherein the plurality of side ports are positioned in a plurality of rows around a circumference of the first outer sidewall.

### BRIEF DESCRIPTION OF SEVERAL VIEWS OF THE DRAWINGS

FIG. 1 is a side elevational view of a medical device according to one embodiment of the present invention;

FIG. 2 is a cross-sectional perspective view of a medical device taken along the line 2-2 of FIG. 1;

FIG. 3 is a schematic representation according to one embodiment of a medical device;

FIG. 4 is a schematic representation according to one embodiment of a medical device;

FIG. 5 is a side elevational view of a medical device according to one embodiment of the present invention;

FIG. 6, is a schematic representation of a medical device according to one embodiment of the present invention;

FIG. 7, is a schematic representation along a cross-sectional longitudinal portion of the medical device in a first position;

FIG. 8 is a schematic representation along a cross-sectional longitudinal portion of the medical device in a second position; and

FIG. 9 is a schematic representation along a cross-sectional longitudinal portion of a medical device according to one embodiment of the present invention.

### DETAILED DESCRIPTION OF THE INVENTION

An exemplary embodiment of the infusion catheter system includes a larger lumen elongated catheter body and a smaller elongated cannula body. The larger catheter includes a plurality of holes, side ports or openings along the length of the body. The smaller cannula body may have a single outlet opening towards its distal end and may be moved laterally within the catheter body. The outlet opening, when positioned relative to a single side port on the catheter body allows therapeutic fluid to flow from the proximal end of the cannula to a designated region of a vein or other vessel.

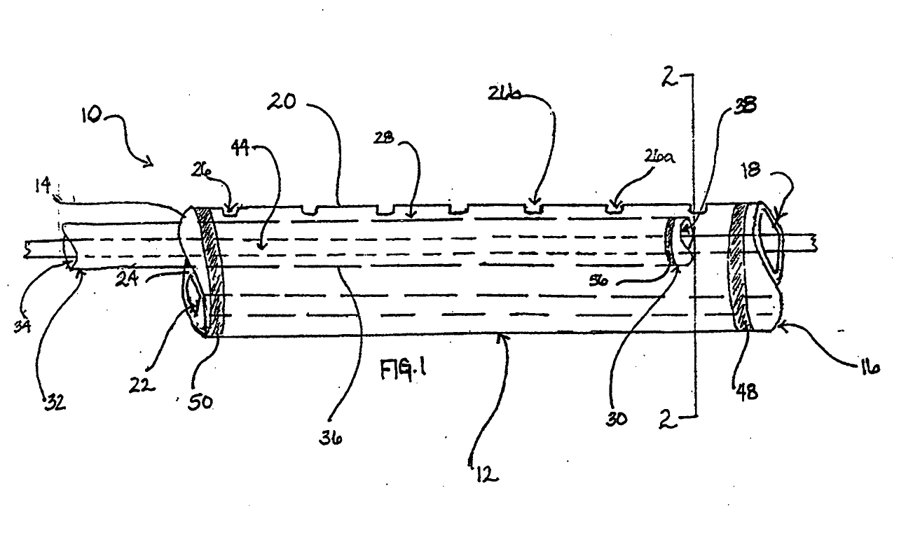

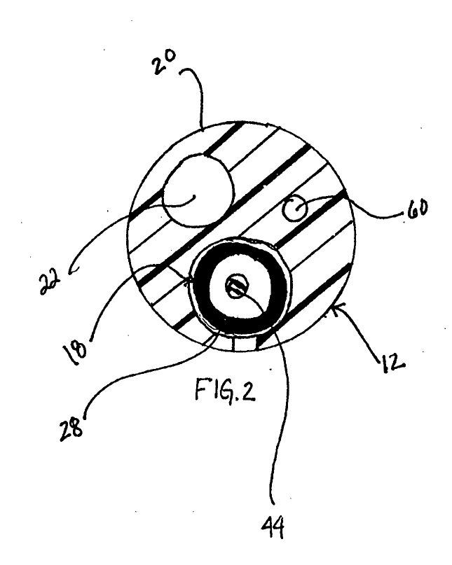

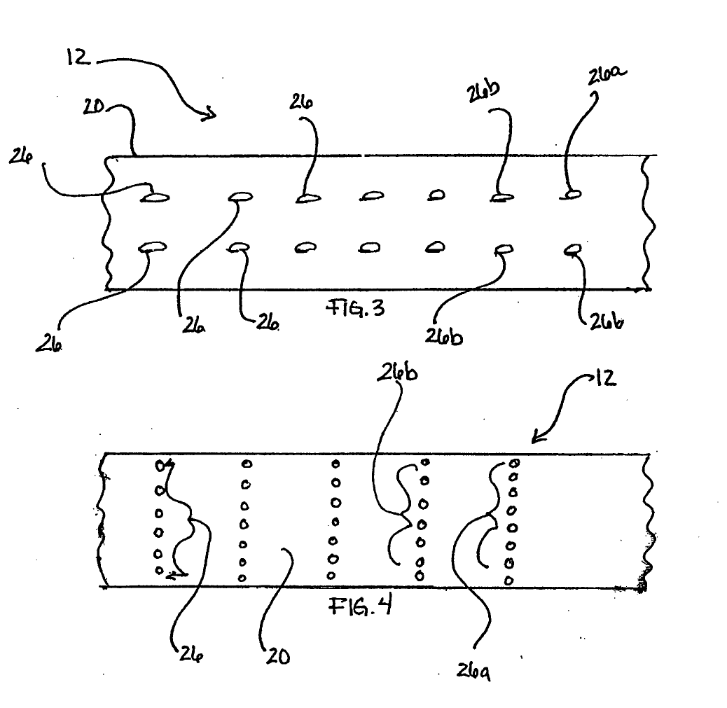

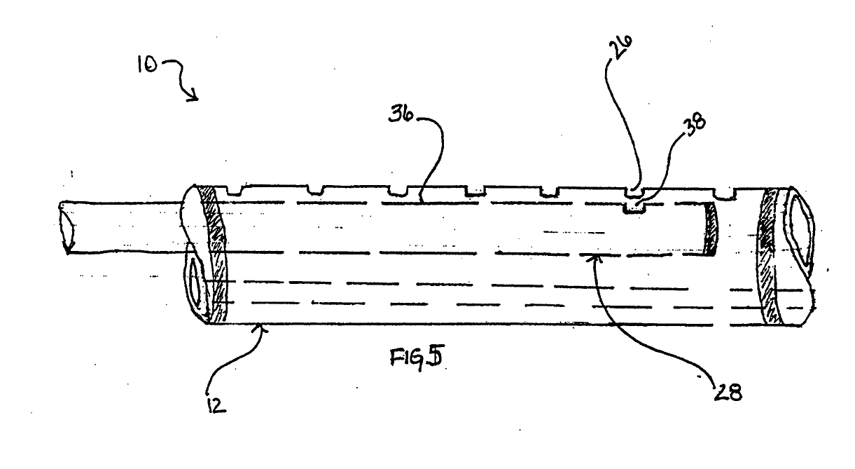

Referring now to FIG. 1, the infusion catheter system 10 includes an elongated catheter body 12 having a first proximal end 14 and a first distal end 16. The catheter body 12 also includes a first lumen 18 which is defined by a first outer sidewall 20, which extends between the distal end 16 and the proximal end 14. As shown in FIGS. 1 and 2, the catheter body 12 may also include an inflation lumen 22 and/or a guidewire lumen 60. The first lumen 18, the inflation lumen 22, and the guidewire lumen 60 are generally separated by at least one inner sidewall 24. The outer sidewall 20 includes a plurality of side ports 26, holes, or openings therethrough. The side ports 26 provide for the delivery of therapeutic fluids to a specified region of an artery, vein or other such vessel. The catheter body 12 may have at least one row of side ports 26 perforating the outer sidewall 20, including at least a first side port 26a, disposed toward the first distal end 16 of the catheter body 12, and a second side port 26b, disposed toward the first proximal end 14 of the catheter body 12. The side ports 26 may be spaced at equal distances along the length of the catheter body 12. However, other configurations, such as positioning the side ports 26 in multiple lines along the length of the catheter body 12 or in rows, circumferentially, are also possible, as shown in FIGS. 3 and 4. The side ports 26 may also be spiraled around the first outer sidewall 20.

The first outer sidewall 20 of the catheter 12 may include side ports 26 that are spaced approximately every centimeter over a 20-30 cm length. The side ports 26 desirably have an inner diameter of approximately 0.2 mm to approximately 5.0 mm. Preferably, the inner diameter is between about 0.25 mm to about 2.0 mm. The catheter body 12 is perforated to form the above-described side ports by using various conventional means such as a drill, a laser, or a punch.

The catheter body 12 may be formed from conventional flexible materials. For example, such materials that may find application for preparing the catheters according to the present invention are polyethylene, polytetrafluorethylene (PTFE), polypropylene, polyethylene terephthalate, nylon and various silicon based polymers. The exterior of the first outer side wall 20 of the catheter body 12 may also contain a hydrophilic coating, e.g., polyvinyl pyrrolidone, polyethylene glycol, polyethylene oxide, or the like to improve the ease of inserting the catheter in to the body of a patient.

The infusion catheter system 10 further includes a smaller elongated cannula body 28 disposed axially within the catheter body 12. The hollow cannula body 28 desirably has a second distal end 30, a second proximal end 32, and an inner lumen 34 defined by a second sidewall 36 extending therebetween. The second sidewall 36 may have no side ports, holes or openings and is generally nonporous or fluid impermeable. An outlet opening 38 may be disposed through the face of the second distal end 30 of the cannula body 28.

Generally, the second side wall 36 of the cannula body 28 does not include side ports and both the second proximal end 32 and the second distal end 30 are both open, placing the outlet opening 38 through the second distal end 30 of the cannula body 28. However, the outlet opening 38 of the cannula body 28 could be through the second side wall 36, rather than through the second distal end 30. In this embodiment, it would generally be contemplated that the second distal end 30 may be closed, as shown in FIG. 5. It is also contemplated that outlet openings 38 in the cannula body 28 could be present in a variety of configurations.

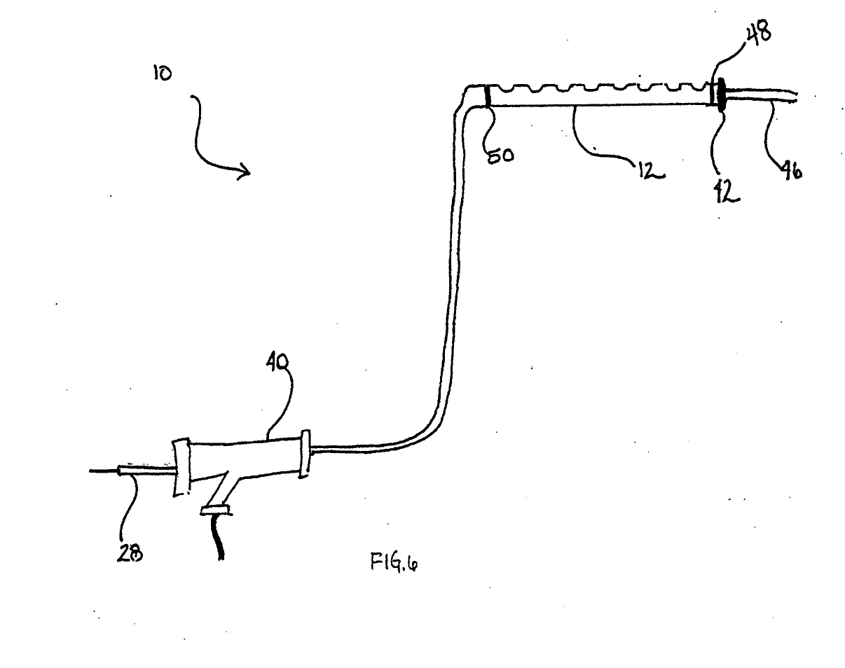

The cannula 28 may also include one mating part of a Luer fitting 40, or similar attachment mechanism, disposed at the second proximal end 32, as shown in FIG. 6. The Luer fitting 40 may be used to accommodate attachment of a syringe or the like which, for this purpose, is formed with the other mating part of the Luer fitting 40 at its attachment end.

For infusion catheters, it is highly desirable to deliver the particular fluid, whether a therapeutic agent or a diagnostic agent, to the particular area of the body for treatment or diagnosis at a controlled and uniform rate of discharge. Not only is it important to control the rate of discharge of fluid to achieve the desired therapeutic effect or diagnostic result, but also it is to avoid or minimize trauma to body tissues due to frictional or shear forces or by too high pressure or volume of fluid. To achieve the desired control and uniform rate of discharge of fluid from the side ports of the catheter, a method is provided that is not dependant on the size of the side port or the change in pressure along the length of the catheter system.

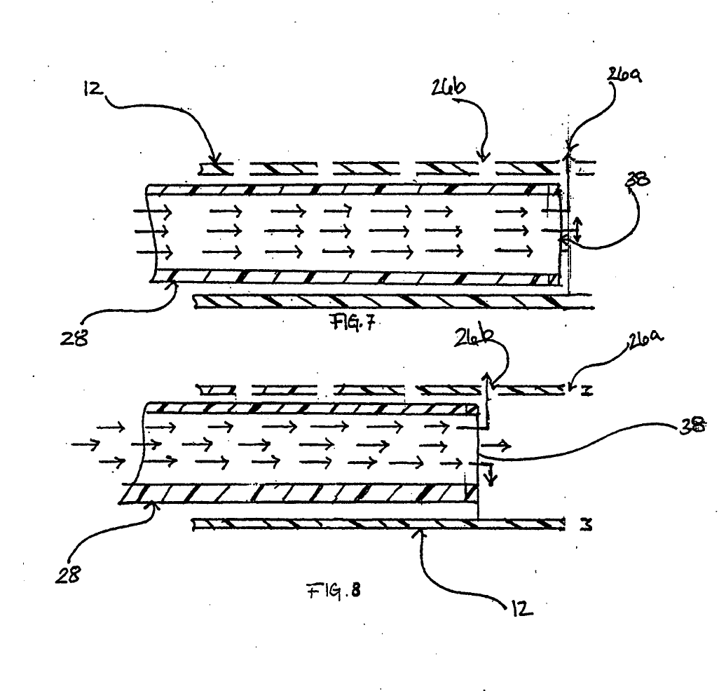

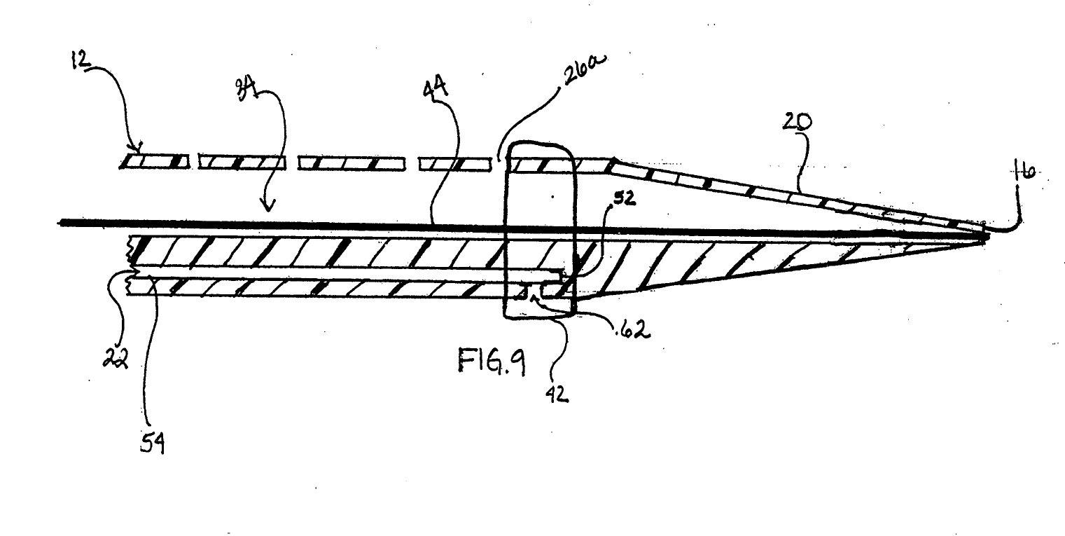

Referring now to FIGS. 7 and 8, the cannula body 28 is movable along a longitudinal direction from a first position to at least a second position. At the first position, shown in FIG. 7, the outlet opening 38 of the cannula body 28 is primarily in fluid communication or adjacent the first side port 26a of the catheter body 12. At the first position, a fluid may be introduced into the inner lumen 34 of the cannula body 28 and is discharged into the vessel through the outlet opening 38 and, consequently, the first side port 26a. When a satisfactory amount of fluid has been discharged at the first position, the cannula body 28 is then manually moved axially to the second position, shown in FIG. 8. At the second position, the outlet opening 38 of the cannula body 28 is primarily in fluid communication or adjacent the second side port 26b of the catheter body 12. Fluid is then introduced into the inner lumen 34 of the cannula body 28 and is subsequently discharged into the vessel through the outlet opening 38 and, consequently, the second side port 26b of the catheter body 12. Desirably, this process may continue as the cannula 28 is telescoped or moved axially toward the proximal end 14 of the catheter body 12, thereby discharging fluid at each desired side port 26 along the length of the catheter body 12. Depending upon the dimensions of catheter body 12 and cannula body 28, a small amount of fluid may be discharged from other sideports 26 that are not primarily in fluid communication with the outlet opening 38. For purposes of maneuverability of the system 10, the system 10 may be designed to have a small space between the outer diameter of the cannula body 28 and the inner diameter of the catheter body 12. The majority of the fluid, however, may be discharged through the primarily selected sideport 26.

As shown in FIG. 6, the catheter system 10 may also include an occlusion device 42, a guidewire 44, a distal radiopaque tip 46, radiopaque markers 48, 50 or other suitable visualization devices to indicate the boundary of the section of the catheter containing the side ports 26.

Referring now to FIG. 9, the occlusion device 42 may generally be disposed at the first distal end 16 of the catheter body 12. The occlusion device 42 may be a balloon extending circumferentially around the outside of the first outer side wall 20. The balloon 42 is generally affixed to the outer side wall 20 with adhesive, heat bonding or other suitable attachment process. The balloon 42 may be selectively inflated by providing fluid under pressure through the inflation lumen 22. In one embodiment, the inflation lumen 22 may include an open proximal end 54, a closed distal end 52, and an inflation port 62 disposed through the side wall of the inflation lumen 22. The inflation port 62 extends from the sidewall of the inflation lumen 22, through the first outer side wall 20 of the catheter body 12 and is in fluid communication with the inside of the balloon 42.

Further, a connector may be mounted at the open proximal end 54 of the inflation lumen 22 and may be attached to a suitable source for supplying pressurized fluid to the balloon 42. Fluid flows from the open proximal end 54, through the inflation lumen 22, through the inflation port 62, and into the balloon 42, inflating the balloon 42 and sealing the flow of blood through the vein. This process occludes the vein and prevents the therapeutic agent from leaving the area to be treated.

The guidewire 44 is generally disposed within the first lumen 18 of the catheter body 12. The guidewire 44 provides for the insertion and advancement of the catheter system 10 into a vein or vessel. In one embodiment, the guidewire is disposed within the inner lumen 34 of the cannula body 28, which is disposed within the first lumen 18 of the catheter body 12. In this embodiment, the first lumen 18 may extend from the first distal end 16 of the catheter 12 to the first proximal end 14 of the catheter 12. The first lumen 18 is preferably open at both the first proximal end 14 and the first distal end 16.

Distal of the first side port 263, the first lumen 18 may be tapered to fit the diameter of the guidewire 44, creating a tight fit between the distal most portion end of the catheter body 12 and the guidewire 44. This arrangement prevents fluid from flowing from the first lumen 18 of the catheter body 12, into the distal end of the vein. One way to taper the catheter body 12 may be to melt the catheter body 12 to an appropriate diameter, as shown in FIG. 9.

Referring again to FIG. 2, the catheter body 12 may include a third, guidewire lumen 60. In this embodiment, the guidewire 44 is threaded through the guidewire lumen 60, rather than the first lumen 18.

Referring to FIG. 6, a radiopaque tip 46 may be molded into the catheter body 12 from a molding composition comprising about 80% tungsten, or other suitable material, by weight. However, any known method for producing a radiopaque tip 46 may be employed. Likewise, the radiopaque markers 48, 50, 56 may be metal and are generally a platinum/iridium (Pt/Ir) metal alloy band. As with the radiopaque tip 46, any known metal or means to render the proximal and distal boundaries radiopaque may be employed.

The radiopaque markers 48, 50, 56 may be disposed at the distal and proximal ends of the catheter body 12 and the cannula body 28. As shown in FIG. 1, the catheter system 10 may include radiopaque markers 48, 50 disposed circumferentially around the first proximal end 16 and the first distal end 14 of the catheter body 12, respectively. The markers 48, 50 indicate the boundary of the portion of the catheter body 12 that includes side ports 26. The catheter system 10 may also include a third radiopaque marker 56 disposed circumferentially around the second distal end 30 of the cannula body 28. The radiopaque markers 48, 50, 56 can be visualized within the body with the use of x-ray, MRI, CT, ultrasound, or other suitable devices.

It is also contemplated that the catheter system 10 may also be visualized without the use of radiopaque markers. In this instance, an x-ray or ultrasound device may be used to visualize the device. In this scenario, separate radiopaque markers may not be needed.

Also, the cannula body 28 may also be “dimpled” or otherwise treated to allow increased visualization of the tip with an ultrasound or other suitable device. Rows of dimples may be placed near the second distal end 30 of the cannula body 28. This process may enhance visualization of the cannula 28 with ultrasound.

A method of treating varicose veins may be used in conjunction with the catheter system 10. Accordingly, to treat an affected varicose vein, the patient's leg is elevated to allow much of the blood in the affected vein to drain into the rest of the body. A small incision is then made in the patient's leg or a small hole is poked into the patient's leg with a needle. The guidewire 44 is then manipulated, using known techniques, through the varicose vein of a patient to reach the region to be treated. An introducer sheath (not shown) is then inserted so that the infusion catheter system 10, as described above, can be threaded onto the guidewire 44 and manipulated into position with the section of the catheter body 12 having the side ports 26 positioned adjacent the region needing treatment. Generally, the catheter system 10 will be threaded onto the guidewire 44 through the inner lumen 34 of the cannula body 28. Once in place, the guidewire 44 will generally extend past the outlet opening 38 of the cannula body 28 and preferably occludes the first distal end 16 of the first lumen 18 of the catheter body 12.

A balloon 42 is then inflated around the catheter body 12 to seal the vein at its distal end. Inflation of the balloon 42 ensures that the therapeutic agent, injected into the vein 58 through the catheter system 10, remains in the affected portion and does not travel to other portions of the body.

Once the catheter system 10 is in place and the affected region of the vein has been isolated by the balloon 42, the cannula body 28 is manually moved into a first position, wherein the outlet opening 38 is positioned adjacent the side port 26 nearest the distal end 16 of the catheter body 12. A sclerosant is loaded into a 20 mL to 30 mL syringe. The syringe is attached to the Luer fitting 40, or other suitable device, on the cannula body 28. Using the syringe, the sclerosant is injected through the inner lumen 34 of the cannula body 28 and is infused through the outlet opening 38, through the first side port 26a, and into the varicose vein. As the vein fills with sclerosant, the cannula 28 is moved toward the proximal end 14 of the catheter body 12 and into the second position, wherein the outlet opening 38 is adjacent a next side port 26b. The movement of the cannula 28 is repeated until the desired portion of the varicose vein is filled with the sclerosant.

After between 1-5 minutes, the vein contracts and becomes fibrosed. The catheter system 10, remains in place while the vein contracts. The entire system 10 is removed upon completion of the procedure.

By enclosing the cannula 28 within the catheter 12 having side ports 26, the therapeutic fluid may be precisely and directly applied to the region to be treated. Moreover, the vein is not agitated by the constant movement of a device within its walls and is filled from the most distal end to the most proximal end of the vein.

Although the invention has been shown and described with respect to preferred embodiments, alterations and modification of the components and methods of the invention may occur to those skilled in the art upon reading and understanding this specification. Accordingly, the present invention is defined by the scope of the claims below and not by the description provided above.

1. An infusion catheter system, comprising:
an elongated catheter body, said catheter body comprising a first distal end, a first proximal end, and at least a first lumen; said first lumen defined by an outer sidewall extending between said distal end and said proximal end; said sidewall having a plurality of side ports therethrough, said side ports including at least a first side port and a second side port disposed longitudinally along said catheter body;
an elongated cannula body disposed within said catheter body and comprising a second distal end, a second proximal end, and a second lumen, said second lumen being defined by a second sidewall extending between said second distal end and second proximal end, and an outlet opening disposed through said cannula; and
wherein said cannula is movable along a longitudinal axis from a first position, wherein said outlet opening is primarily in fluid communication with said first side port, and a second position, wherein said outlet opening is primarily in fluid communication with said second side port.

2. The infusion catheter system of claim 1, wherein said plurality of side ports each have an inner diameter of approximately 0.2 mm to approximately 2.0 mm.

3. The infusion catheter system of claim 2, wherein said plurality of side ports each have an inner diameter of approximately 0.8 mm.

4. The infusion catheter system of claim 1, wherein said plurality of side ports each have an inner diameter of approximately 0.8 mm and wherein said side ports are longitudinally disposed along said side wall approximately every one cm over a 20 cm length.

5. An infusion catheter system of claim 1, wherein said side ports are disposed longitudinally along said catheter body for at least 20 cm, and wherein said side ports are disposed approximately 1 cm from one another.

6. The infusion catheter system of claim 1, wherein said catheter body further comprises a first radiopaque band, said band disposed circumferentially around said catheter body at said first distal end.

7. The infusion catheter system of claim 6, wherein said catheter body further comprises a second radiopaque band, said band disposed circumferentially around said catheter body at said first proximal end; and
wherein said first radiopaque band and said second radiopaque band delineate a boundary of said catheter body having said side ports.

8. The infusion catheter system of claim 1, wherein said system further comprises an occlusion device disposed distal of said first distal end.

9. The infusion catheter system of claim 1, wherein said elongated cannula body is characterized by a lack of outlet openings in said second side wall and wherein said outlet opening is disposed through said second distal end of said cannula body.

10. The infusion catheter system of claim 1, wherein said outlet opening is disposed through said second sidewall, adjacent said second distal end.

11. A method of sclerosing a vein comprising the steps of:
inserting an infusion catheter system into a vein, said catheter system comprising:
an elongated catheter body, said catheter comprising a first distal end, a first proximal end, and at least a first lumen; said first lumen defined by a first outer sidewall extending between said first distal end and said first proximal end; said first outer sidewall having a plurality of side ports therethrough, said side ports including at least a first side port and a second side port disposed longitudinally along said catheter body;
an elongated cannula body disposed within said catheter body and comprising a second distal end, a second proximal end, and a second lumen, said second lumen being defined by a second sidewall extending between said second distal end and second proximal end, and an outlet opening disposed through said second distal end; and
wherein said cannula is movable along a longitudinal axis from at least a first position, wherein said outlet opening is primarily in fluid communication with said first side port, and a second position, wherein said outlet opening is primarily in fluid communication with said second side port;
injecting a sclerosant into said cannula body, when said cannula body is in said first position, said sclerosant thereby passing through said first side port and sclerosing a first portion of said vein;
moving said cannula body from said first position to said second position;
injecting said sclerosant into said cannula body when said cannula body is in said second position, said sclerosant thereby passing through said second side port and sclerosing a second portion of said vein.

12. The method of claim 11, further comprising inserting a guidewire into said vein and manipulating said guidewire through said vein to reach a portion to be treated.

13. The method of claim 12, wherein said catheter system further comprises an occlusion balloon, said balloon being inflated around said first distal end of said catheter body and before said sclerosant is injected into said cannula body.

14. The method of claim 11, wherein said catheter system further comprises at least a first radiopaque marker disposed adjacent said first distal end of said catheter body and defining a distal boundary of a portion of said catheter body having said side ports.

15. The method of claim 14, wherein said catheter system further comprises a second radiopaque marker disposed proximal from said first radiopaque marker and further defining a proximal boundary of said portion of said catheter body having said side ports.

16. The method of claim 15, further comprising positioning said first radiopaque marker and said second radiopaque marker, to overlap an area of said vein to be treated and positioning said cannula body relative to said first radiopaque marker and said second radiopaque marker.

17. The method of claim 11, wherein said plurality of side ports are arranged in a single row and are disposed along said longitudinal axis of said infusion catheter system.

18. The method of claim 11, wherein said plurality of side ports are arranged in two rows along a longitudinal axis of said catheter body.

19. The method of claim 11, wherein said plurality of side ports are positioned in a plurality of rows around a circumference of said first side wall.
